(19) United States
(12) Patent Application Publication
(10) Pub. No.: US 2021/0403226 A1
(43) Pub. Date: Dec. 30, 2021

(54) FLIP-TOP VIAL WITH SHAKE-OUT STRIP DISPENSER AND MOLDED DESICCANT

(71) Applicants: James B. Babcock, Phoenix, MD (US); Christopher Donald Webb, Stewartstown, PA (US)

(72) Inventors: James B. Babcock, Phoenix, MD (US); Christopher Donald Webb, Stewartstown, PA (US)

(21) Appl. No.: 17/358,192

(22) Filed: Jun. 25, 2021

Related U.S. Application Data
(60) Provisional application No. 63/046,232, filed on Jun. 30, 2020.

(51) Int. Cl. B65D 83/08; B65D 81/26; B65D 43/16; B65D 25/14

(52) U.S. Cl. CPC B65D 83/08; B65D 25/14; B65D 43/162; B65D 81/266

(57) ABSTRACT

A vial can include a container having a sidewall, a bottom, and a top, which may include a vial opening. The vial can include a lid that attaches to the top and that seals the opening. The vial can include a first dispenser insert that attaches proximate to the top. The vial can include an inner lining along an interior face of the sidewall. The inner lining can include a desiccant material.

### CROSS-REFERENCE TO RELATED APPLICATION(S)

The present application derives priority from U.S. provisional application Ser. No. 63/046,232 filed 30 Jun. 2020.

### BACKGROUND OF THE INVENTION

### 1. Field of the Invention

The present invention relates to chemical test equipment in general, and more specifically to test strip storage containers.

### 2. Description of the Background

Test strips are well-known media to test for the presence of particular chemical species in the air, smokestacks, water, and the like. These test strips are single-use disposables and/or consumables and they are generally provided in bulk containers: typically a plastic vial with removable or flip-top cap. The test strips are subject to spoilage and must be stored in an environment that is as moisture free as possible. Conventional test sample containers employ detached desiccant materials to attempt to lower moisture levels and thereby preserve and maintain the integrity of the samples. For example, desiccant pillows are commonly added to the vials. Thus, tens or hundreds of test strips may be stored in a single vial along with a desiccant pillow in a fairly unorganized way. These separate desiccant materials and strips often move within the test container and damage the strips. Worse yet, the use of such vials creates a risk of spilling the entire contents of the vial when one is only trying to extract a single test strip. The spilled test strips can be contaminated, in which case they would have to be discarded. Moreover, the desiccant pillow may get wet. In this case even if the spilled test strips can be salvaged, moisture bearing air will most likely be introduced into the container and sealed therein upon closure, and detrimentally absorbed by the test strips.

Therefore, what is needed is a strip dispenser with integral desiccant and a mechanism to dispense/eject a single test strip that is simple in design and inexpensive to manufacture. What is further needed is a strip dispenser that is a manual device that reproducibly presents a single test strip to the user upon shaking without exposing the remaining test strips.

### SUMMARY OF THE INVENTION

Accordingly, it is an object of the present invention to provide an improved test strip dispensing vial with integral desiccant and a shaker mechanism to dispense/eject a single test strip.

Another object is to provide a test strip dispensing vial as above that is simple in design and inexpensive to manufacture.

Yet another object is the provide a strip dispenser that reliably and consistently presents a single test strip to the user upon shaking without exposing the remaining test strips.

These and other features and benefits are achieved with an improved test-strip dispenser vial formed as a closed-bottom cylindrical enclosure with open top and a flip-top lid flexibly hinged to the rim of the open top to seal the vial opening. The radius of the flip-top lid exceeds that of the dispenser vial so as to close overtop. The dispenser vial is of known axial length L₁. A cylindrical desiccant liner of known axial length L₂ is inserted into the dispenser vial, where L₁>L₂. The shake-through dispenser is a disc-shaped member of thickness L₃ with a constricted central aperture, and seats atop the cylindrical desiccant liner, occupying the remaining space such that L₁≈L₂+L₃. This way, when the flip-top lid is closed overtop the dispenser vial its inner surface abuts and seals the aperture of the shake-through dispenser.

For a more complete understanding of the invention, its objects and advantages, refer to the remaining specification and to the accompanying drawings.

### BRIEF DESCRIPTION OF THE DRAWINGS

Other objects, features, and advantages of the present invention will become more apparent from the following detailed description of the preferred embodiments and certain modifications thereof when taken together with the accompanying drawings in which:

FIG. 1 shows a top perspective view of an example vial receiving a testing strip, according to embodiments of the invention.

FIG. 2 is a side cross-section of the vial of FIG. 1.

FIG. 3 is a top perspective view of an example dispenser insert, according to embodiments of this disclosure.

FIG. 4 is a side cross-section of the example dispenser insert of FIG. 3.

FIG. 5 is a top perspective view of an example dispenser insert, according to embodiments of this disclosure.

FIG. 6 is a side cross-section of the example dispenser insert of FIG. 5.

FIG. 7 is a top perspective view of an example dispenser insert, according to embodiments of this disclosure.

FIG. 8 is a side cross-section of the example dispenser insert of FIG. 7.

FIG. 9 is a top perspective view of an example dispenser insert, according to embodiments of this disclosure.

FIG. 10 is a side cross-section of the example dispenser insert of FIG. 8.

FIG. 11 is a top perspective view of an example dispenser insert, according to embodiments of this disclosure.

FIG. 12 is a side cross-section of the example dispenser insert of FIG. 11.

FIG. 13 is a top perspective view of an example dispenser insert, according to embodiments of this disclosure.

FIG. 14 is a side cross-section of the example dispenser insert of FIG. 13.

FIG. 15 is a top perspective view of an example dispenser insert, according to embodiments of this disclosure.

FIG. 16 is a side cross-section of the example dispenser insert of FIG. 15.

FIG. 17 is a top perspective view of an example dispenser insert, according to embodiments of this disclosure.

FIG. 18 is a side cross-section of the example dispenser insert of FIG. 17.

FIG. 19 shows a top perspective view of example vial having a desiccant inner lining, with a testing strip and without a dispenser insert, according to embodiments of this disclosure.

### DETAILED DESCRIPTION OF THE PREFERRED EMBODIMENTS

The present invention is simple, easy to assemble test strip dispenser vial with integral desiccant and a shake mechanism to dispense/eject a single test strip without exposing the remaining test strips. In a preferred embodiment test-strip dispenser comprises a vial formed as a closed-bottom frusto-conical or cylindrical enclosure with open mouth and a flip-top lid flexibly hinged to the rim of the open mouth. The flip-top lid closes and detent-locks overtop the mouth. A cylindrical desiccant liner is inserted into the dispenser vial, and a conforming disc-shaped shaker partition with a central aperture seats atop the cylindrical desiccant liner. When the flip-top lid is closed its inner surface abuts and seals the aperture of the shaker partition. Thus, the test strip dispenser can improve the integrity and preservation of samples collected over conventional test sample storage devices.

The following lists the numbered features shown in the drawings:

100 Vial
110 Container
111 Container
120 Sidewall
121 Sidewall
130 Bottom
131 Bottom
140 Mouth
141 Mouth
142 Hinge
143 Hinge
151 Vial opening
160 Lid
161 Lid
173 raised collar
181 Inner lining
200 Test strip
210 Testing panel
300 Dispenser insert
301 Top surface
302 Inner edge
303 Dispenser aperture
304 Outer edge
310 Dispenser insert
311 Top surface
312 Inner edge
313 Dispenser aperture
314 Outer edge
315 Ribs
316 Hinges
320 Dispenser insert
321 Top surface
322 Inner edge
323 Dispenser aperture
324 Outer edge
325 Ribs
326 Hinges
330 Dispenser insert
331 Top surface
332 Inner edge
333 Dispenser aperture
334 Outer edge
335 Outer lip
337 Tapered edge
340 Dispenser insert
341 Top surface
342 Inner edge
343 Dispenser aperture
344 Outer edge
350 Dispenser insert
351 Top surface
352 Inner edge
353 Dispenser aperture
354 Outer edge
355 Outer lip
357 Tapered edge
359 Aperture surface
360 Dispenser insert
361 Top surface
362 Inner edge
363 Dispenser aperture
364 Outer edge
365 Outer lip
366 Curved transition
367 Tapered edge
368 Curved transition
369 Aperture surface
370 Dispenser insert
371 Top surface
372 Inner edge
373 Dispenser aperture
374 Outer edge
375 Outer lip
376 Curved transition
377 Tapered edge
378 Curved transition
379 Aperture surface

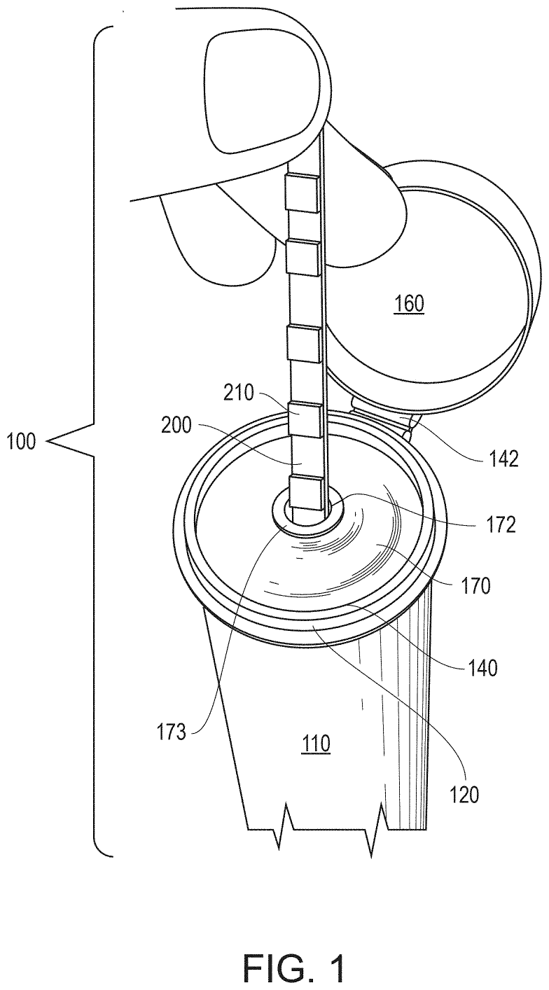

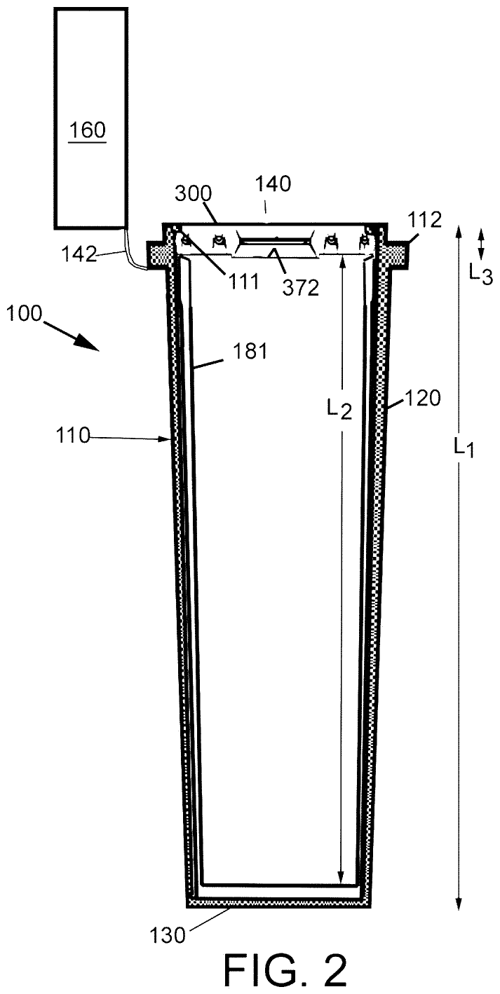

FIG. 1 shows a top perspective view and FIG. 2 a cross-section of an exemplary test strip dispenser vial 100 with inner lining 181 according to an embodiment of the invention. Vial 100 includes container 110 having a round cylindrical or frusto-conical sidewall 120, closed-bottom 130, and open mouth 140. Vial 100 includes lid 160 that closes-over the mouth 140. Lid 160 is preferably attached to the rims of mouth 140 by flexible hinge 142. A peripheral outer lip 112 rings the sidewall 120 of vial 100 externally and offset slightly below mouth 140 to engage the lid 160. Vial 100 includes a dispenser insert 300 that slides into the open mouth 140. Dispenser insert 300 preferably has a central dispenser aperture 372 for dispensing single test strips, though one skilled in the art will understand that aperture 372 need not be centered, but rather can be closer to one of the sidewalls. Vial 100 includes a cylindrical inner lining 181 (FIG. 2) interiorly conforming to cylindrical sidewall 120 and, optionally, the closed-bottom 130. The inner lining 181 includes a desiccant material. Vial mouth 140 can be sealed by lid 161 with or without a dispenser insert 300 attached within container 110.

As shown in FIG. 1, test strip 200 includes various reagent-testing panels 210. During manufacture, inner lining 181 is inserted, then test strips 200 are inserted in bulk into vial 100 through open mouth 140. Next, dispenser insert 170 is inserted and seated atop inner lining 181. The diameter of the central dispenser aperture 172 of dispenser insert 170 conforms to but is very slightly larger than the width of test strip 200 such that only a single test strip 200 can be removed from vial 100 through dispenser aperture 172. The collective test strips 200 can be sealed in vial 100 by pivoting lid 160 on flexible hinge 142 and pressing hard until lid 160 press-fit over open mouth 140. One skilled in the art will understand that vial 100 may take different shapes, e.g., rectangles, polyhedrons, and/or other suitable geometric forms, but cylindrical or frusto-conical are preferred.

Dispenser insert 170 is a disc-shaped member suitable for insertion in container 110. Dispenser insert 300 preferably forms a pressure or friction fit within mouth 140 above inner lining 181 keeping the inner lining 181 fixed there beneath. In some embodiments sidewall 120 may have fixation features such as interior ribs or threading and dispenser insert 300 may have exterior corresponding ribs or threading such that dispenser insert 300 can engage or screw into container 110. In a preferred embodiment the dispenser insert 300 rests on an inner lip formed by insertable inner lining 181 and is retained by an outer lip 111 formed at the mouth 140 of container 110. Toward this end, the diameter of the flip-top lid 160 very slightly exceeds that of the dispenser vial 100 so as to close overtop. The dispenser vial 100 is of known axial length L₁. A conforming (cylindrical or frusto-conical) inner lining 181 of known axial length L₂ is inserted into the dispenser vial, where L₁>L₂. The disc-shaped dispenser insert 300 has a thickness L₃ and seats atop the cylindrical inner lining 181, occupying the remaining space such that L₁≈L₂+L₃. This way, when the flip-top lid 160 is closed overtop the container 110 its inner surface abuts and seals the aperture 172 of the dispenser insert 300.

Dispenser insert 300 is inserted into a container 110 of vial 100 to enclose the top 140. Dispenser insert 300 can have a variety of geometries with a variety of dimensions. For example, dispenser insert 300 can have a dispenser aperture 303 that is substantially circular, ellipsoidal, or rectangular polygonal shape. Dispenser aperture 303 invariably has a maximum dimension that is less than a dimension of the vial opening of vial 100 and greater than a dimension of insertable testing strip 200. For example, a width or diameter of dispenser aperture 303 can be less than a width or diameter of the vial opening of vial 100 and greater than the width or diameter of insertable testing strip 200. In embodiments where dispenser aperture 303 forms a substantially ellipsoidal or circular shape, a major axis or diameter of the dispenser aperture 303 can be only slightly larger than the width of an insertable test strip 200, such that two or more stacked test strips 200 could not concurrently fit through dispenser aperture 172, but a single test strip 200 could. Given the foregoing constraints, once a test strip 200 is inserted into dispenser aperture 303 it becomes difficult or highly unlikely that test strip 200 will unintentionally exit dispenser aperture 303.

Maintaining environmental conditions, such as atmospheric moisture within a vial, can be beneficial for preserving test samples and maintaining their integrity. A variety of components can be included with vials for maintaining environmental conditions within vial containers, according to embodiments of this disclosure. As seen in FIG. 1 dispenser insert 300 may have a raised collar 173 surrounding dispenser aperture 172 to form a better seal against closed lid 161.

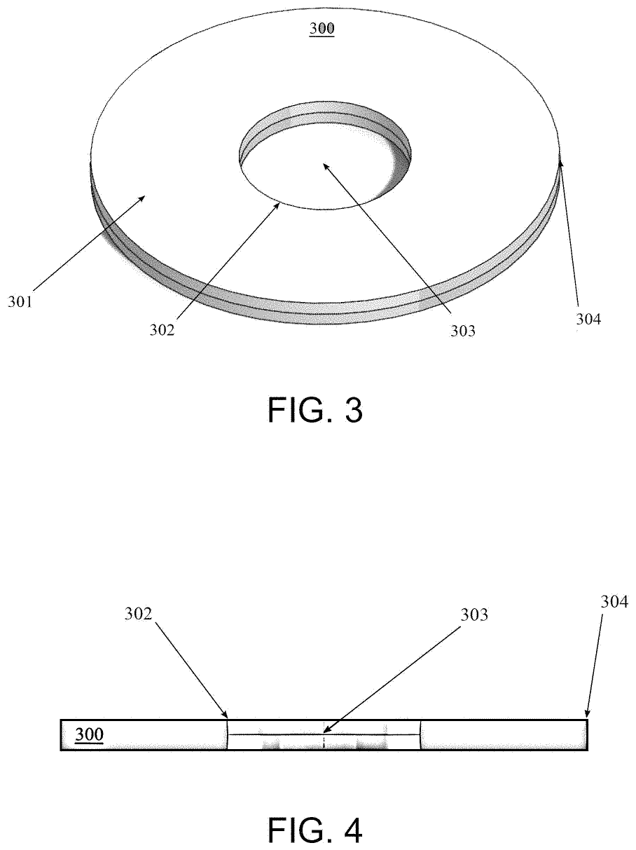

FIG. 3 shows a top perspective view and FIG. 4 shows a cross section view of a dispenser insert 300, according to an embodiment of the invention. Dispenser insert 300 can have a flat top surface 301 and be formed from solid materials. Inner edge 302 of dispenser aperture 303 can be formed in a substantially symmetrical and concentric shape with respect to the outer edge 304 of dispenser insert 300. Top surface 301 can be substantially symmetrical with the bottom surface of dispenser insert 300. Manufacturing of dispenser inserts having flat top and bottom surfaces, such as dispenser insert 300, can be more efficiently automated than dispenser inserts having substantial protrusions from their top and bottom surfaces. In operation, dispenser insert 300 can be minimally deformable such that it forms a relatively stiff enclosure with other components with sidewall 121 of container 111.

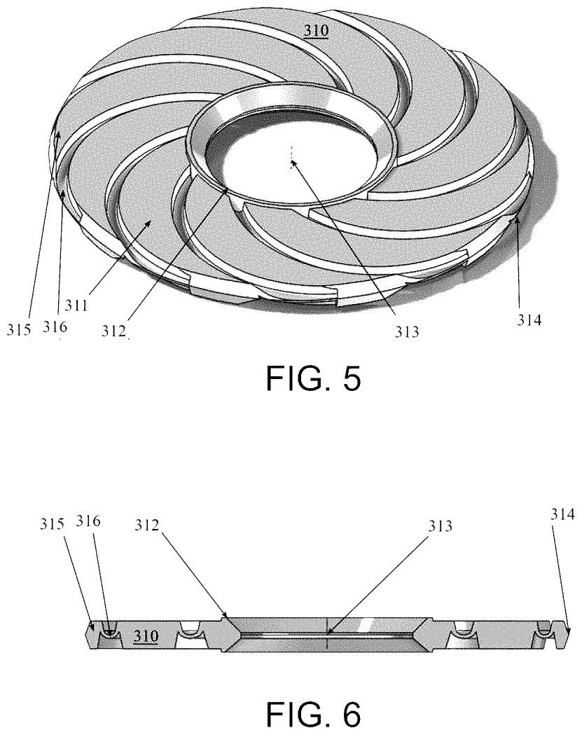

FIGS. 5-18 show additional example dispenser inserts 310, 320, 330, 340, 350, 360, 370 according to the invention. In FIG. 5, dispenser insert 310 can have a top surface 311 and be formed from solid materials. Inner edge 312 of dispenser aperture 313 can be formed in a substantially symmetrical and concentric shape with respect to the outer edge 314 of dispenser insert 310. Inner edge 312 can be tapered as shown to enable easier test strip feeding and dispensing. Dispenser insert 310 can include ribs 315 and hinges 316 in an arrangement such as a spiral arrangement or any other arrangement suitable for the purposes of this disclosure. In operation, dispenser insert 310 can be deformable such that top surface 311 or bottom surface can deform flatly or conically within sidewalls 121 of container 111, either inward or outward from an opening of container 111. For example, hinges 316 can compress variably along their length to result in a reduced circumference of dispenser insert 310. In some embodiments, dispenser insert 310 can fit an opening of container 111 such that deformation is not apparent and top surface 311 is relatively flat. For example, hinges 316 may compress, while remaining portions of insert 310 remain substantially un-deformed.

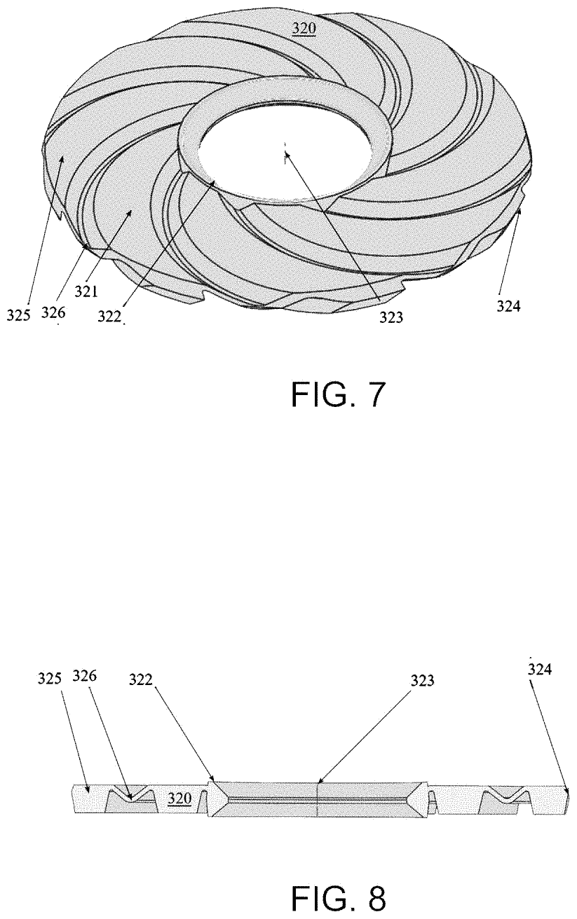

FIG. 7 shows a top perspective view and FIG. 8 shows a cross section view of another example dispenser insert 320, according to an embodiment of the invention. Dispenser insert 320 can have a top surface 321 and be formed from solid materials. Inner edge 322 of dispenser aperture 323 can be formed in a substantially symmetrical and concentric shape with respect to the outer edge 324 of dispenser insert 320. Inner edge 322 can be tapered as shown to enable easier test strip feeding. Dispenser insert 320 can include ribs 325 and hinges 326 in an arrangement such as a spiral arrangement or any other arrangement suitable for the purposes of this disclosure. In operation, dispenser insert 320 can be deformable such that top surface 321 or bottom surface can deform flatly or conically within sidewalls 121 of container 111, either inward or outward from an opening of container 111. For example, hinges 326 can compress variably along their length to result in a reduced circumference of dispenser insert 320. Dispenser insert 320 can have relatively wider hinges 326 than hinges 316 of dispenser insert 310 enabling a greater degree of hinge compression and overall insert deformity than dispenser insert 310. In some embodiments, dispenser insert 320 can fit an opening of container 111 such that deformation is not apparent and top surface 321 is relatively flat. For example, hinges 326 may compress, while remaining portions of insert 320 remain substantially un-deformed.

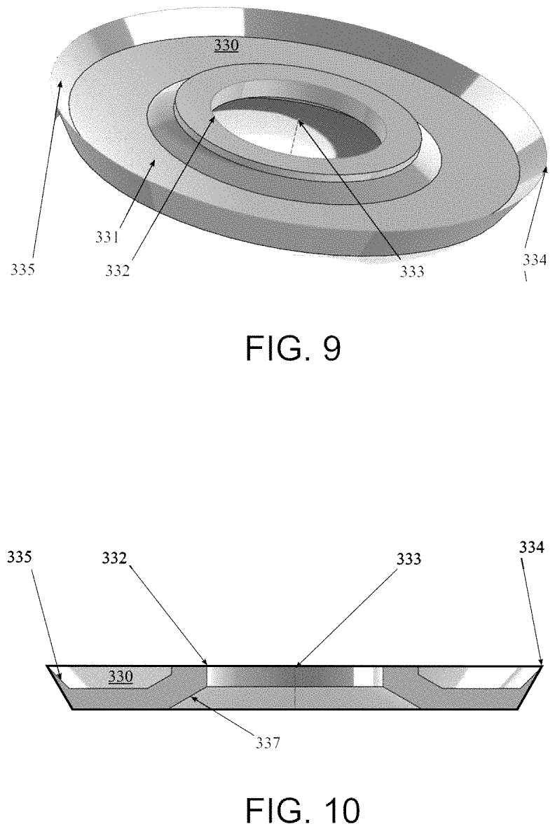

FIG. 9 shows a top perspective view and FIG. 10 shows a cross section view of another example dispenser insert 330, according to an embodiment of this invention. Dispenser insert 330 can have a top surface 331 and be formed from solid materials. Top surface 331 can be asymmetrical with the bottom surface of dispenser insert 330. Inner edge 332 of dispenser aperture 333 can be formed in a substantially symmetrical and concentric shape with respect to the outer edge 334 of dispenser insert 330. Inner edge 332 can be substantially vertical and then tapered as shown at tapered edge 337. Tapered edge 337 can enable test strips 200 to more easily dispense from dispenser aperture 333 from inside of container 111. Dispenser insert 330 can be deformable to form a compression fit within sidewalls 131 of container 111. For example, outer edge 334 may be an edge of outer lip 335 extending outward from the bottom surface of dispenser insert 330. In operation, outer lip 335 may be highly deformable depending on the relative thickness of materials forming outer lip 335. For example outer lip 335 may be relatively more deformable at relatively thin sections closer to outer edge 334 than relatively thick sections of outer lip 335 closer to the bottom surface of dispenser insert 330. Outer lip 335 may compress when fit into sidewalls 131 of container 111 to form a compression fit within container 111.

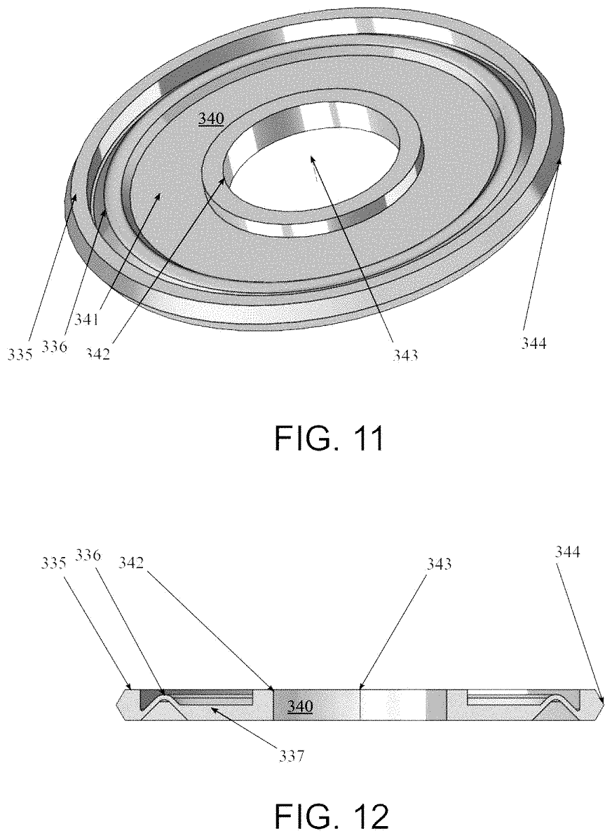

FIG. 11 shows a top perspective view and FIG. 12 shows a cross section view of another example dispenser insert 340, according to an embodiment of the invention. Dispenser insert 340 can have a top surface 341 and be formed from solid materials. Top surface 341 can be asymmetrical with the bottom surface of dispenser insert 340. Inner edge 342 of dispenser aperture 343 can be formed in a substantially symmetrical and concentric shape with respect to the outer edge 344 of dispenser insert 340. Dispenser insert 340 can include outer rib 335 and hinge 336 in an arrangement such as a circumferential arrangement with respect to inner edge 342, or any other arrangement suitable for the purposes of this disclosure. Inner edge 342 can be substantially vertical. In operation, dispenser insert 340 can be deformable to form a compression fit within sidewalls 131 of container 111. For example, outer rib 335 can compress hinge 336 to reduce the overall circumference of dispenser insert 340, while remaining portions of insert 340 remain substantially un-deformed.

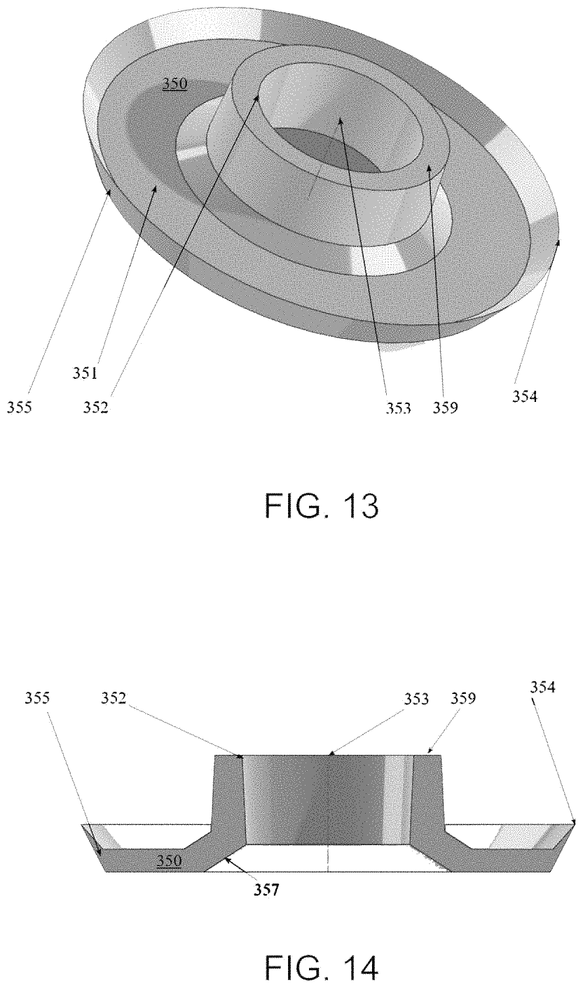

FIG. 13 shows a top perspective view and FIG. 14 shows a cross section view of another example dispenser insert 350, according to an embodiment of the invention. Dispenser insert 350 can have a top surface 351 and be formed from solid materials. Top surface 351 can be asymmetrical with the bottom surface of dispenser insert 350. Dispenser aperture 353 can extend outward from container such that aperture surface 359 is substantially flush with an inner surface of flip-top lid 160 when the flip-top lid 160 is closed. In the closed position, flip-top lid 160 can form a second seal between aperture surface 359 and flip-top lid 160 in addition to the first seal created by flip-top lid 160 and sidewalls 131 of container 111. This second seal can further preserve test strips 200 and also prevent loosening of the components of dispenser insert 350, including test strips 200 during transportation. Inner edge 352 of dispenser aperture 353 can be formed in a substantially symmetrical and concentric shape with respect to the outer edge 354 of dispenser insert 350. Inner edge 352 can be substantially vertical and then tapered as shown at tapered edge 357. Tapered edge 357 can enable test strips 200 to more easily dispense from dispenser aperture 353 from inside of container 111. Dispenser insert 350 can be deformable to form a compression fit within sidewalls 131 of container 111. For example, outer edge 354 may be an edge of outer lip 355 extending outward from the bottom surface of dispenser insert 330. In operation, outer lip 355 may be highly deformable depending on the relative thickness of materials forming outer lip 355. For example, outer lip 355 may be relatively more deformable at relatively thin sections closer to outer edge 354 than relatively thick sections of outer lip 355 closer to the bottom surface of dispenser insert 350. Outer lip 355 may compress when fit into sidewalls 131 of container 111 to form a compression fit within container 111. In addition, the extended outer lip 355 serves to prevent the dispenser insert 350 from becoming unseated during use and handling, and even if this does occur, the outer lip 355 facilitates reseating because each time that the lid 160 is reclosed it pushes the dispenser insert 350 back into place.

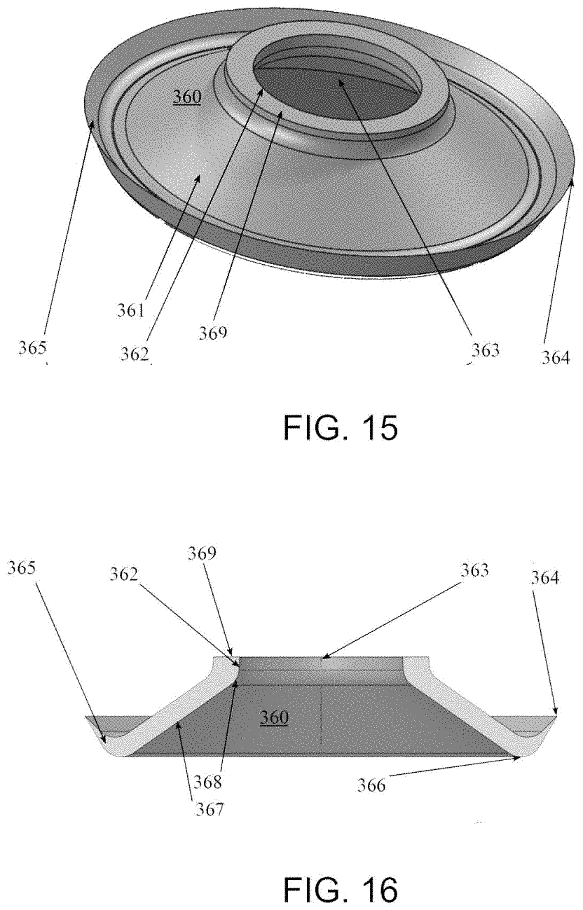

FIG. 15 shows a top perspective view and FIG. 16 shows a cross section view of another example dispenser insert 360, according to an embodiment of the invention. Dispenser insert 360 can have a top surface 361 and be formed from solid materials. Top surface 361 can be asymmetrical with the bottom surface of dispenser insert 360. Dispenser aperture 363 can extend outward from container such that aperture surface 369 is substantially flush with an inner surface of flip-top lid 160 when the flip-top lid 160 is closed. In the closed position, flip-top lid 160 can form a second seal between aperture surface 369 and flip-top lid 160 in addition to the first seal created by flip-top lid 160 and sidewalls 131 of container 111. This second seal can further preserve test strips 200 and also prevent loosening of the components of dispenser insert 360, including test strips 200 during transportation. Inner edge 362 of dispenser aperture 363 can be formed in a substantially symmetrical and concentric shape with respect to the outer edge 364 of dispenser insert 360. Inner edge 362 can be substantially vertical and then tapered as shown at tapered edge 367. Tapered edge 367 can enable test strips 200 to more easily dispense from dispenser aperture 363 from inside of container 111. Inner edge 362 can transition to tapered edge 367 at curved transition 368 to further reduce friction when dispensing test strips 200. Dispenser insert 360 can be deformable to form a compression fit within sidewalls 131 of container 111. For example, outer edge 364 may be an edge of outer lip 365 extending outward from the bottom surface of dispenser insert 360 along curved transition 366. In operation, outer lip 365 may be highly deformable depending on the relative thickness of materials forming outer lip 365. For example, outer lip 365 may be relatively more deformable at relatively thin sections closer to outer edge 364 than relatively thick sections of outer lip 365 closer to the bottom surface of dispenser insert 360. Outer lip 365 may compress when fit into sidewalls 131 of container 111 to form a compression fit within container 111.

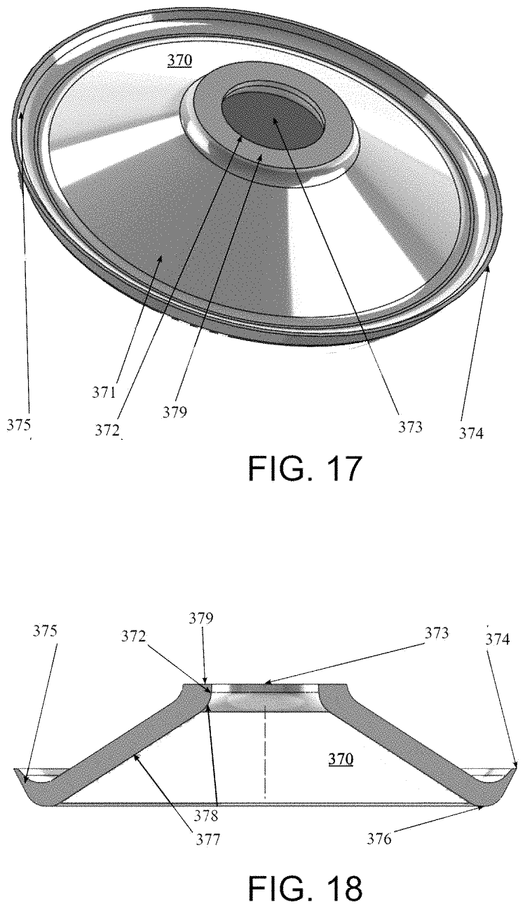

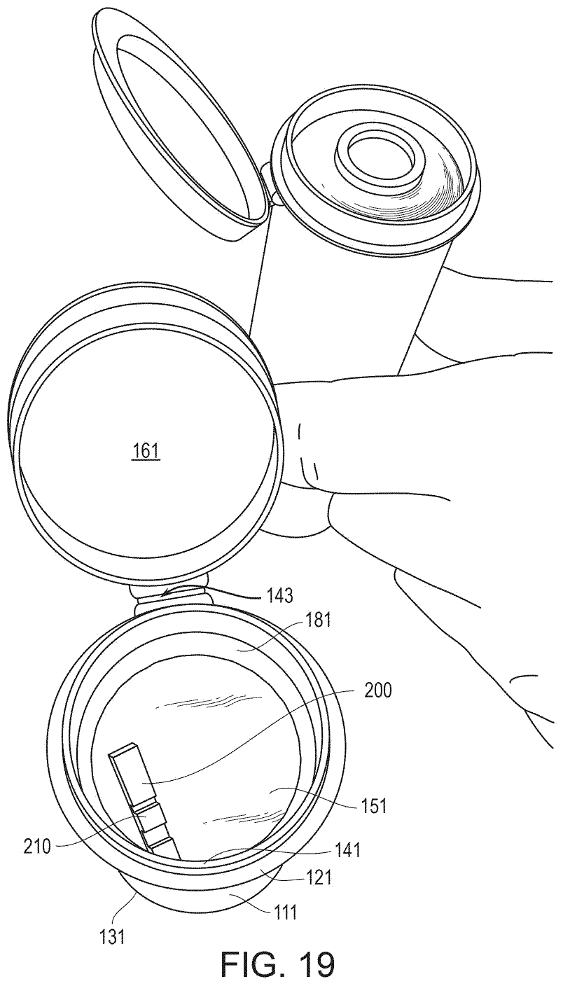

FIG. 17 shows a top perspective view and FIG. 18 shows a cross section view of another example dispenser insert 370, according to an embodiment of the invention. Dispenser insert 370 can have a relatively larger dispenser aperture 373 than dispenser aperture 363 of dispenser insert 360 to reduce light and moisture intrusion into container 111. Dispenser insert 370 can have a top surface 371 and be formed from solid materials. Top surface 371 can be asymmetrical with the bottom surface of dispenser insert 370. Dispenser aperture 373 can extend outward from container such that aperture surface 379 is substantially flush with an inner surface of flip-top lid 160 when the flip-top lid 160 is closed. In the closed position, flip-top lid 160 can form a second seal between aperture surface 379 and flip-top lid 160 in addition to the first seal created by flip-top lid 160 and sidewalls 131 of container 111. This second seal can further preserve test strips 200 and also prevent loosening of the components of dispenser insert 360, including test strips 200 during transportation. Inner edge 372 of dispenser aperture 373 can be formed in a substantially symmetrical and concentric shape with respect to the outer edge 374 of dispenser insert 370. Inner edge 372 can be substantially vertical and then tapered as shown at tapered edge 377. Tapered edge 377 can enable test strips 200 to more easily dispense from dispenser aperture 373 from inside of container 111. Inner edge 374 can transition to tapered edge 377 at curved transition 378 to further reduce friction when dispensing test strips 200. Dispenser insert 370 can be deformable to form a compression fit within sidewalls 131 of container 111. For example, outer edge 374 may be an edge of outer lip 375 extending outward from the bottom surface of dispenser insert 370 along curved transition 376. In operation, outer lip 375 may be highly deformable depending on the relative thickness of materials forming outer lip 375. For example, outer lip 375 may be relatively more deformable at relatively thin sections closer to outer edge 374 than relatively thick sections of outer lip 375 closer to the bottom surface of dispenser insert 370. Outer lip 375 may compress when fit into sidewalls 131 of container 111 to form a compression fit within container 111.

FIG. 19 shows a top perspective view of an example vial 100 having a desiccant inner lining 181, with a testing strip 200 and without a dispenser insert 300, according to embodiments of the invention. All or part of inner lining 181 can be formed of a desiccant material to induce or sustain a state of dryness within vial 100. Inner lining 181 can include any desiccant material suitable for the purposes of this disclosure, such as silica, activated charcoal, calcium sulfate, calcium chloride, and/or molecular sieves. In a preferred embodiment, inner lining 181 is molded from a desiccant entrained polymer into a cylindrical shape that has an exterior surface substantially conforming to the interior surface of the vial 110 and is press-fit into position where it is held sufficiently snugly to prevent its unintended disengagement.

Embodiments of this disclosure can employ any mechanism for attaching inner lining 181 to sidewall 121 that is suitable for the purposes of this disclosure. For example, in some embodiments, inner lining 181 can be formed as an inner sleeve or inner cup that substantially conforms to the dimensions of sidewall 121 and/or bottom 131 and thereby attaches via friction. This way inner lining 181 can be removable from sidewall 121 of container 111 and additional replacement inner linings 181 can be included if the initial inner lining 181 is no longer effective. In some embodiments, inner lining 181 can be molded, glued, or fused to sidewall 121 and/or bottom 131. Desiccant inner lining 181 may be initially molded, inserted and allowed to harden, such that expansion characteristics of them vial 110 shrink-fits the inner lining 181. In still a further embodiment, inner lining 181 and sidewall 121 can be formed as a single molded component by a molding process.

Inner linings 181 can be formed from a variety of materials, including a variety of desiccant materials. In some embodiments, a vial 101 can include an initial inner lining 181 and a variety of replacement inner linings 181, where the initial inner lining 181 can include a particular desiccant material and the replacement inner linings 181 can include a different desiccant materials. Varieties of desiccant materials may be beneficial for storing samples at different times that requiring different dryness conditions. As an example, an entrained polymer for desiccant inner lining 181 may be about 45 wt. % of polyvinyl alcohol, about 5 wt. % of glycerin and about 55 wt. % of propylene maleic anhydride. Polyvinyl alcohol is mixed with glycerin until the polyvinyl alcohol is evenly wetted by the glycerin. Propylene maleic anhydride is then blended with the mixture. The resulting blend is then fed to an extruder to react the mixture. The extruder is operated at about 400 to about 450° with a residence time of between about 20-45 seconds. The melt is extruded in a film and, subsequently, ground into a fine powder. This fine powder is mixed with silica gel desiccant. The powder-silica gel mixture is then extruded into a film. The silica gel in the film absorbs moisture.

Another example of a desiccant-entrained plastic vial assembly for effervescent tablets is shown in U.S. Pat. No. 7,413,083 to Belfance et al. issued Aug. 19, 2008.

In general, components of embodiments of this disclosure, such as container 111, bottom 131, sidewall 121, mouth 141, hinge 143, lid 161, inner lining 181, and dispenser insert 300 can be formed from any materials suitable for the purposes of this disclosure. For example, components of embodiments can be formed from metals, such as titanium, magnesium, tungsten, aluminum, steel; plastics; and resins. Components of embodiments of this disclosure can be manufactured according to any manufacturing technique suitable for the purpose of this disclosure, such as extrusion, blow molding, casting, and/or additive manufacturing processes. Components of embodiments of this disclosure can be attached or otherwise joined according to any attachment mechanisms suitable for the purposes of this disclosure. For example, components may be screwed, latched, clasped, clamped, stapled, fused, bonded, glued, welded, and/or compression fit together.

Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept. It is to be understood, therefore, that the invention may be practiced otherwise than as specifically set forth in the appended claims.

We claim:

1. A vial, comprising:
a container having a sidewall, a bottom, and a top having a vial opening;
a lid that attaches to the top and seals the vial opening;
a dessicant liner lining of an interior face of the sidewall formed of a first desiccant material;
a first dispenser insert inserted into the vial opening of said container and seated proximate the vial opening atop the dessicant liner.

2. The vial of claim 1, wherein a flexible hinge attaches the lid to the top.

3. The vial of claim 1, wherein the first dispenser insert comprises a dispenser opening that has a dimension less than a dimension of the vial opening and greater than a width of an insertable testing strip.

4. The vial of claim 1 wherein the first dispenser insert comprises a dispenser opening configured to concurrently dispense no more than one insertable testing strip.

5. The vial of claim 1, wherein the first dispenser insert attaches within the container.

6. The vial of claim 1, wherein the inner lining and the sidewall are formed as a single molded component by a molding process.

7. The vial of claim 1, wherein the inner lining is removable from the sidewall of the container.

8. The vial of claim 1, wherein the inner lining comprising the first desiccant material extends to an interior face of the bottom.

9. The vial of claim 1, further comprising at least one second dispenser insert, wherein the at least one second dispenser insert comprises a dispenser opening having a dimension that is different from a dimension of the first dispenser insert.

10. The vial of claim 1, further comprising a replacement inner lining comprising the first desiccant material.

11. The vial of claim 1, further comprising a replacement inner lining comprising a second desiccant material that is different from the first desiccant material.

12. The vial of claim 1, wherein said first desiccant inner lining comprises polyvinyl alcohol, glycerin and propylene maleic anhydride.

13. The vial of claim 1, wherein said first desiccant inner lining comprises about 45 wt % of polyvinyl alcohol, about 5 wt. % of glycerin, and about 55 wt % of propylene maleic anhydride.

14. The vial of claim 1, wherein said lid has a flat surface, and said first dispenser insert has a central aperture surroundeed by a raised lip that seals against the flat surface of the lid

15. A test-strip dispenser, comprising:
a plurality of test strips each having at least one reagent pad, all of said plurality of test strips having a constant length and width;
a dispenser vial formed as a round closed-bottom enclosure with open mouth;
a flip-top lid attached to the rim of the dispenser vial mouth by a flexible hinge;
a round desiccant liner inserted into the dispenser vial;
a disc-shaped shaker partition having an aperture seated atop the desiccant liner, the aperture of said shaker partition having maximum width slightly greater than the common width of said plurality of test strips.

16. The test strip dispenser of claim 15, wherein said lid has a flat surface, and said first dispenser insert has a central aperture surroundeed by a raised lip configured to seal against the flat surface of the lid.

17. The test strip dispenser of claim 15, wherein said dispenser vial is formed as a frusto-conical enclosure.

18. The test strip dispenser of claim 15, wherein said dispenser vial is formed as a cylindrical enclosure.

19. The test strip dispenser of claim 15, wherein said dispenser vial has an interior length L₁, said shaker partition has a thickness L₃, and said plurality of test strips all have a length L₂ where L₂≤L₁−L₃.
